(19) United States
(12) Patent Application Publication
(10) Pub. No.: US 2015/0023888 A1
(43) Pub. Date: Jan. 22, 2015

(54) Method of Using Dihydroergotamine for Pre-emptive Treatment of Migraine

(71) Applicant: MAP Pharmaceuticals, Inc., Irvine, CA (US)

(72) Inventors: ROBERT O. COOK, Hillsborough, NJ (US); PAUL L. DURHAM, Nixa, MO (US)

(73) Assignee: MAP Pharmaceuticals, Inc.

(21) Appl. No.: 14/503,027

(22) Filed: Sep. 30, 2014

Related U.S. Application Data
(63) Continuation of application No. 12/584,395, filed on Sep. 3, 2009.
(60) Provisional application No. 61/191,349, filed on Sep. 5, 2008.
(60) Provisional application No. 61/191,189, filed on Sep. 5, 2008.

(51) Int. Cl. A61K 31/4985; A61K 9/00; A61K 47/06

(52) U.S. Cl. CPC A61K 31/4985; A61K 47/06; A61K 9/008
USPC 424/45; 514/250

(57) ABSTRACT

The invention is drawn to a novel, early interventional use of a pharmaceutical composition wherein administration of said pharmaceutical composition pre-empts the onset of migraine headaches, preferably anytime in the migraine cycle prior to pain onset. The pharmaceutical composition preferably is administered via inhalation in order to provide therapy to the subject in a timely fashion, ideally in a non-clinical setting. The pharmaceutical composition may also be administered to a subject at risk for stroke.

### CROSS REFERENCE TO RELATED CASES

This application is a continuation application to U.S. patent application Ser. No. 12/584,395, filed on Sep. 3, 2009, which claims the benefit of U.S. Provisional Application Ser. No. 61/191,349 filed on Sep. 5, 2008 and U.S. Provisional Application Ser. No. 61/191,189 filed on Sep. 5, 2008, the contents of which are herein incorporated by reference in their entirety.

### FIELD OF THE INVENTION

The invention provides for compositions and methods for pre-emptive treatment of migraine headache, and also for the reducing the incidence of stroke.

### BACKGROUND OF THE INVENTION

Migraine is one of the most common neurological disorders and comprises periodic attacks of headache and nausea and a variety of other symptoms. Although considerable progress has been made, the pathophysiology of migraine is far from understood. A number of observations have, however, pointed to the involvement of the “calcitonin gene related peptide” (CGRP). Migraine headaches involve the activation of the neuronal trigeminal system and the dilation of intracranial, extracerebral blood vessels. CGRP is located in the neurons in trigeminal ganglia, and the CGRP levels are released from this region during a migraine attack, which in turn causes the vasodilatation observed. It is therefore conceivable that inhibiting the dilation of the cranial blood vessels caused by CGRP might possibly give rise to a new treatment for migraine headaches. Medicaments widely used for treating migraine are the so-called “triptans”, e.g. sumatriptan and zolmitriptan. These compounds derive their activity against migraine from their vasoconstrictor properties and presumably their inhibition of the release of the neuropeptide calcitonin gene related peptide (CGRP) (Ferrari, M. D., Saxena, P. R. (1995), 5-HT1 receptors in migraine pathophysiology and treatment, Eur. J. Neurology, 2, 5-21; Johnson, K. W., Phebus, L. A., Cohen, M. L. (1998), Serotonin in migraine: Theories, animal models and emerging therapies, Progress in Drug Research, vol. 51, 220-244), assuming that the levels thereof are raised during a migraine attack (Edvinsson, L., Goadsby, P. J. (1994), Neuropeptides in migraine and cluster headache, Cephalgia, 14(5), 320-327). A new approach for the treatment of migraine is the use of CGRP antagonists (Doods, H., Hallermayer, G., Wu, D., Entzeroth, M., Rudolf, K., Engel, W., Eberlein, W. (2000), Pharmacological profile of BIBN4096BS, the first selective small molecule CGRP antagonist, Br. J. Pharmacol., 129, 420-423). This approach however only serves to respond to CGRP once released, and does not prevent release from occurring.

Migraine headaches afflict 10-20% of the U.S. population, with an estimated loss of 64 million workdays annually. Migraine headache is characterized by pulsating head pain that is episodic, unilateral or bilateral, lasting from 4 to 72 hours and often associated with nausea, vomiting and hypersensitivity to light and/or sound. When accompanied by premonitory symptoms, such as visual, sensory, speech or motor symptoms, the headache is referred to as “migraine with aura,” formerly known as classic migraine. When not accompanied by such symptoms, the headache is referred to as “migraine without aura,” formerly known as common migraine. Both types evidence a strong genetic component, and both are three times more common in women than men. The precise etiology of migraine has yet to be determined. It has been theorized that persons prone to migraine have a reduced threshold for neuronal excitability, possibly due to reduced activity of the inhibitory neurotransmitter y-aminobutyric acid (GABA). GABA normally inhibits the activity of the neurotransmitters serotonin (5-HT) and glutamate, both of which appear to be involved in migraine attacks. The excitatory neurotransmitter glutamate is implicated in an electrical phenomenon called cortical spreading depression, which can initiate a migraine attack, while serotonin is implicated in vascular changes that occur as the migraine progresses.

It has been suggested that cortical spreading depression (CSD) underlies migraine visual aura. CSD is characterized by a short burst of intense depolarization in the occipital cortex, followed by a wave of neuronal silence and diminished evoked potentials that advance anteriorly across the surface of the cerebral cortex. Enhanced excitability of the occipital-cortex neurons has been proposed as the basis for CSD. The visual cortex may have a lower threshold for excitability and therefore is most prone to CSD. It has been suggested that mitochondrial disorders, magnesium deficiency and abnormality of presynaptic calcium channels may be responsible for neuronal hyperexcitability (Welch, Pathogenesis of Migraine, Seminars in Neurobiol., 17:4, 1997). During a spreading depression event, profound ionic perturbations occur, which include interstitial acidification, extracellular potassium accumulation and redistribution of sodium and chloride ions to intracellular compartments. In addition, prolonged glial swelling occurs as a homeostatic response to altered ionic extracellular fluid composition, and interstitial neurotransmitter and fatty acid accumulation. Studies have shown that furosemide inhibits regenerative cortical spreading depression in anaesthetized cats (Read et al, Cephalagia, 17:826, 1997).

A study of eighty-five patients with refractory transformed migraine type of chronic daily headache (CDH) concluded that acute headache exacerbations responded to specific anti-migraine agents such as ergotamine, dihydroergotamine (DHE) and sumatriptan, and that addition of agents such as acetazolamide and furosemide, after diagnosis of increased intracranial pressure, resulted in better control of symptoms (Mathew et al. Neurology 46:1226-1230, May 1996). The authors note that these results suggest a possible link between migraine and idiopathic intracranial hypertension that needs further research. It has also been reported that furosemide appeared to abort prolonged visual auras in two migraine patients. The author speculated that furosemide may act to inhibit CSD activity (Rozen, Neurology, 55:732-3, 2000).

Drug therapy is tailored to the severity and frequency of migraine headaches. For occasional attacks, abortive treatment may be indicated, but for attacks occurring two or more times per month, or when attacks greatly impact the patient's daily life, prophylactic therapy may be indicated. Serotonin receptor agonists, such as sumatriptan, have been prescribed for abortive therapy. These are thought to constrict dilated arteries of the brain, thereby alleviating the associated pain. Side effects associated with the use of serotonin receptor agonists include tingling, dizziness, warm-hot sensations and injection-site reactions. Intravenous administration is contraindicated due to the potential for coronary vasospasms.

Drugs used for prophylactic treatment of migraine include andrenergic beta-blockers such as propranolol, calcium channel blockers, and low-dose anti-epileptic drugs. In particular, anti-epileptic drugs that increase brain levels of GABA, either by increasing GABA synthesis or reducing its breakdown, appear to be effective in preventing migraine in certain individuals. In some patients, tricyclic analgesics, such as amitriptline, can be effective. NMDA receptor antagonists, which act at one of the glutamate receptor subtypes in the brain, inhibit CSD. Drugs or substances currently believed to function as weak NMDA receptor antagonists include dextromethorphan, magnesium, and ketamine. Intravenous magnesium has been successfully used to abort migraine attacks.

Currently available drugs to alleviate the pain associated with migraines have modest or limited efficacy and are associated with various debilitating side effects. Thus, better therapies are needed for the management of migraines.

Antimigraine drugs are well-known. See, e.g., U.S. Pat. Nos. 4,650,810, 4,914,125, 4,916,125, 4,994,483, 5,021,428, 5,200,413, 5,242,949, 5,248,684, 5,273,759, 5,317,103, 5,364,863, 5,399,574, 5,434,154, 5,441,969, 5,464,864, 5,466,699, 5,468,768, 5,491,148 and 5,494,910. Antimigraine drugs most commonly used in treatment of migraine fall into the following groups: beta-blocking agents, calcium channel blocking agents, antidepressants, selective 5-HT₁ agonists (sumatriptan), sedatives, local anesthetics, adrenergic blocking agents and mixtures of these.

The success of triptans in the treatment of migraine is limited. Such drugs (e.g., rizatriptan) show only a 60-70% efficacy. Some antimigraine drugs may have direct, or indirect effect on the 5-hydroxytryptamine (5-HT) receptor system. The 5-HT receptor system is a potent intracranial vasoconstrictor. 5-HT receptors are presently delineated into three major subclassifications—5-HT₁, 5-HT₂, and 5-HT₃—each of which may also be heterogeneous. The receptor mediates vasoconstriction in the carotid vascular bed and thereby modifies blood flow therein. The various classes of compounds have been proposed as 5-HT agonists or antagonists for therapeutic use of migraine, but these have not always been specific to a particular type of 5-HT receptor.

Dihydroergotamine (DHE) is indicated for acute treatment of migraine and cluster headache. It binds with a high affinity for 5-HT_{1A}, 5-HT_{1B}, 5-HT_{1B}, 5-HT_{2A}, 5-HT_{2C} receptors, noradrenaline a2A, a2B, and a receptors, and dopamine D2L and D3 receptors. The therapeutic activity of DHE is generally attributed to the agonist effect at 5-HT_{1D} receptors.

Cutler (US Patent Publication Number 2004/0191178) recites administering dihydroergotamine as a sublingual spray or aerosol for the treatment of migraine but admits that the mode of action is unknown. Cutler also submits that administration through the sublingual mucosa is desirable in that lower doses are used.

Management of migraine is complicated by the lack of a single therapy that is effective in all patients having the same migraine type and by the need to select either an abortive or prophylactic method of treatment for these migraines. Further complications involve the current use of drugs that cause dependence with extended use. Another important consideration is that the more effective antimigraine agents in current use produce severe use-limiting side effects with long-term usage.

At present the United States Federal Drug Agency (FDA) has authorized such medications and drugs for prophylactic usage to prevent unanticipated migraine, however the FDA may consider such practices and self-administration of such pharmaceutical compositions to result in incorrect and unsupervised dosing, as well as greater risk of unpredictable side-effects upon other organs, particularly the heart. In the future, the FDA may use its authority to introduce a more restrictive set of regulations under which such therapies may be administered.

A key step in the pre-emptive treatment of a migraine would be to enable self-administration therapy of a medication or drug during the prodrome phase, which is easily recognizable to the subject and therefore does not involve prophylaxis, potential over-use of medications and drugs, and inappropriate exposure of the subject's body and organs to repeated drug insult and resulting side effects. In particular, administration of a medication or drug during the prodrome phase may thereby reduce or significantly decrease the onset of cortical spreading depression (CSD) thus preventing the progression of migraine.

In addition, it has recently been observed that the risk for stroke (cerebral aneurysm) appears to be greater in migraneures than in the non-migraneur population. This can be explained by repeated migraine insults to the cerebrovascular tissue over time that lead to pathology. By preventing the insults, one can anticipate less progression to stroke pathology.

Thus, there is a need for a safe and effective drug for the treatment of migraine and related disorders which can be used to pre-emptively prevent a migraine during the prodrome phase that minimizes side effects preferably by allowing for the use of lower doses of the therapeutic compound.

### SUMMARY OF THE INVENTION

The invention provides systems and methods for delivering a pharmaceutical formulation to the brain of a subject having a migraine, at risk for having a migraine, any time in the migraine cycle prior to the onset of pain, or at risk for having a stroke. In addition, the invention disclosed herein may also be drawn to the treatment of dyslexia. Administration is preferably via inhalation in order to provide therapy to the subject in a timely fashion, ideally in a non-clinical setting. In clinical settings, a subject would experience in a delay between the initiation of the migraine cycle and receipt of a therapeutic agent, since subjects tend to present at the clinic once pain is moderate to severe and migraine is fully developed.

In one embodiment, the invention provides a method for the pre-emptive suppression of migraine in a migraineur, the method comprising the steps of: (i) providing a subject having a prodrome migraine episode; (ii) administering a pharmaceutical formulation to said individual, the pharmaceutical formulation comprising a pharmaceutical compound and a carrier, wherein, when present in the tissue of the subject, the pharmaceutical compound upregulates expression and activity of mitogen-activated protein kinase phosphatase (MAP kinase phosphatase). In a preferred embodiment the pharmaceutical compound is selected from the group comprising neurotransmitters such as acetylcholine, adrenaline, noradrenaline, glutamate, aspartate, glycine, taurine, dopamine, 5-hydroxytryptamine, melatonin, y-aminobutyric acid, histamine, bradykinin, endorphins, enkephalins, a-MSH, neurokinins, oxytocin, substance P, a glucocorticoid, a toll-like receptor-4 ligand, and an ergot alkaloid, and an ACE inhibitor.

In a preferred embodiment the ergot alkaloid is selected from the group consisting of an ergoline and an ergotamine. In a more preferred embodiment, ergoline is selected from the group consisting of cabergoline, ergine, isoergine, methergine, hydergine, ergonovine, lisuride, lysergic acid, metergoline, methysergide, nicergoline, and pergolide. In another more preferred embodiment, ergotamine is selected from the group consisting of bromocriptine, dihydroergocornine, dihydroergocristine, dihydroergocryptine, dihydroergotamine, dihydroergotoxine, ergotamine, ergocristine, ergocornine, ergocryptine, ergovaline, and ergotinine In a yet more preferred embodiment, ergotamine is dihydroergotamine. In a most preferred embodiment dihydroergotamine is the mesylate salt.

In another embodiment, the method results in a protective upregulation of MAP kinase phosphatase activity. In one preferred embodiment, the protective upregulation of MAP kinase phosphatase activity is acute. In another alternative embodiment, the method results in the long-term protective upregulation of MAP kinase phosphatase activity.

In another embodiment, the invention provides a method for the pre-emptive suppression of migraine in a migraineur, the method comprising the steps of: (i) providing a subject having a prodrome migraine episode; (ii) administering a pharmaceutical formulation to said individual, the pharmaceutical formulation comprising an ergot alkaloid and a carrier; (iii) measuring the relative intensity of the migraine pain at regular intervals using a International Headache Society Pain Scoring or other suitable pain or migraine questionnaire wherein the migraineur reports a decrease in the relative intensity of the pain compared with relative intensity of pain measured during a migraine episode prior to administrating the pharmaceutical formulation, the method resulting in a pre-emptive suppression of the migraine.

In a preferred embodiment the ergot alkaloid is selected from the group consisting of an ergoline and an ergotamine. In a more preferred embodiment, ergoline is selected from the group consisting of ergonovine, lisuride, lysergic acid, metergoline, methysergide, nicergoline, and pergolide. In another more preferred embodiment, ergotamine is selected from the group consisting of bromocriptine, dihydroergocornine, dihydroergocristine, dihydroergocryptine, dihydroergotamine, dihydroergotoxine, and ergotamine. In a yet more preferred embodiment, ergotamine is dihydroergotamine. In a most preferred embodiment dihydroergotamine is the mesylate salt.

In one embodiment the method results in a decrease of the subject's score by at least 5% percent compared with a baseline measurement made prior to administrating the pharmaceutical formulation.

In another embodiment the pharmaceutical formulation is administered to the nasal epithelium. In another embodiment the pharmaceutical formulation is administered to the lung epithelium. In another embodiment the pharmaceutical formulation is administered to the bronchial epithelium. In another embodiment the pharmaceutical formulation is administered to the alveolar epithelium.

In another embodiment the method results in a decrease in the subject's circulating von Willebrand factor by at least 5 percent compared with a baseline measurement made prior to administrating the pharmaceutical formulation.

In another embodiment the electrical activity in the brain of the subject is decreased by at least 5 percent compared with a baseline measurement made prior to administrating the pharmaceutical formulation. In a preferred embodiment the electrical activity in the brain of the subject is measured using an electroencephalogram.

In another embodiment the transport of inflammatory molecules between neural tissues in the brain of the subject is decreased by at least 5 percent. In a more preferred embodiment the transport of inflammatory molecules is through a gap junction. In a yet more preferred embodiment the gap junction comprises connexin 26. In another yet more preferred embodiment connexin 26 binds to or is reduced by DHE. In another preferred embodiment binding of DHE to connexin 26 or DHE's reduction of connexin 26 level reduces the transport of inflammatory molecules. In a more preferred embodiment binding of DHE to connexin 26 or DHE's reduction of connexin 26 level reduces the transport of potassium. In an alternative more preferred embodiment said method results in a decrease in the subject's cortical levels of neuropeptides. In another more preferred embodiment the neuropeptide mediates pain in the subject's cortex. In a most preferred embodiment the neuropeptide is selected from the group consisting of CGRP, substance P, and neurokinin A. In another most preferred embodiment, the decrease in the subject's cortical levels of neuropeptide is decreased by at least 5 percent. In another alternative embodiment said method results in an increase of the subject's anti-inflammatory compounds in the subject's cortex. In a more preferred embodiment said subject's anti-inflammatory compounds comprise phosphates.

In another alternative embodiment, said method results in a decrease of the subject's connexin 26 in glia in the subject's cortex. In a more preferred embodiment the decrease in the subject's connexin 26 is decreased by at least 5 percent.

In another alternative embodiment said method results in an increase of the subject's mitogen-activated protein kinase phosphatase (MKP) levels in the subject's cortex. In a more preferred embodiment, said method results in a decrease of the subject's p38 mitogen-activated protein kinase 14 (p38MAPK) levels in the subject's cortex.

In another alternative embodiment the invention provides a method for reducing the incidence of stroke in a migraineur, the method comprising the steps of: (i) providing a subject having a prodrome migraine episode; (ii) administering a pharmaceutical formulation to said individual, the pharmaceutical formulation comprising an ergot alkaloid and a carrier; (iii) measuring the relative intensity of the migraine pain at regular intervals using a International Headache Society Pain Scoring or other suitable pain or migraine questionnaire wherein the migraineur reports a decrease in the relative intensity of the pain compared with relative intensity of pain measured during a migraine episode prior to administrating the pharmaceutical formulation, the method reducing the incidence of stroke.

In one embodiment the method further comprising the steps of (iv) subjecting said subject to electromagnetic stimulation of the subject's cortex; (v) measuring the subject's ability to correctly identify a series of at least three consecutive symbols before and during electromagnetic stimulation; (vi) comparing the results from before and during electromagnetic stimulation to generate a score, wherein a decrease in the subject's ability to correctly identify a series of at least three consecutive symbols correlates with the efficacy of the pharmaceutical formulation to pre-emptively suppress CSD.

In a preferred embodiment the ergot alkaloid is selected from the group consisting of an ergoline and an ergotamine. In a more preferred embodiment, ergoline is selected from the group consisting of ergonovine, lisuride, lysergic acid, metergoline, methysergide, nicergoline, and pergolide. In another more preferred embodiment, ergotamine is selected from the group consisting of bromocriptine, dihydroergocornine, dihydroergocristine, dihydroergocryptine, dihydroergotamine, dihydroergotoxine, and ergotamine. In a yet more preferred embodiment, ergotamine is dihydroergotamine. In a most preferred embodiment dihydroergotamine is the mesylate salt.

In one embodiment the method results in a decrease of the subject's score by at least 5% percent compared with a baseline measurement made prior to administrating the pharmaceutical formulation.

In another embodiment the pharmaceutical formulation is administered to the nasal epithelium. In another embodiment the pharmaceutical formulation is administered to the lung epithelium. In another embodiment the pharmaceutical formulation is administered to the bronchial epithelium. In another embodiment the pharmaceutical formulation is administered to the alveolar epithelium.

In another embodiment the method results in a decrease in the subject's circulating von Willebrand factor by at least 5 percent compared with a baseline measurement made prior to administrating the pharmaceutical formulation.

In another embodiment the electrical activity in the brain of the subject is decreased by at least 5 percent compared with a baseline measurement made prior to administrating the pharmaceutical formulation. In a preferred embodiment the electrical activity in the brain of the subject is measured using an electroencephalogram.

In another embodiment the transport of inflammatory molecules between neural tissues in the brain of the subject is decreased by at least 5 percent. In a more preferred embodiment the transport of inflammatory molecules is through a gap junction. In a yet more preferred embodiment the gap junction comprises connexin 26. In another yet more preferred embodiment connexin 26 binds to or is reduced by DHE. In another preferred embodiment binding of DHE to connexin 26 or DHE's reduction of connexin 26 level reduces the transport of inflammatory molecules. In a more preferred embodiment binding of DHE to connexin 26 or DHE's reduction of connexin 26 level reduces the transport of potassium. In an alternative more preferred embodiment said method results in a decrease in the subject's cortical levels of neuropeptides. In another more preferred embodiment the neuropeptide mediates pain in the subject's cortex. In a most preferred embodiment the neuropeptide is selected from the group consisting of CGRP, substance P, and neurokinin A. In another most preferred embodiment, the decrease in the subject's cortical levels of neuropeptide is decreased by at least 5 percent. In another alternative embodiment said method results in an increase of the subject's anti-inflammatory compounds in the subject's cortex. In a more preferred embodiment said subject's anti-inflammatory compounds comprise phosphates.

In another alternative embodiment, said method results in a decrease of the subject's connexin 26 in glia in the subject's cortex. In a more preferred embodiment the decrease in the subject's connexin 26 is decreased by at least 5 percent.

In another alternative embodiment said method results in an increase of the subject's mitogen-activated protein kinase phosphatase (MKP) levels in the subject's cortex. In a more preferred embodiment, said method results in a decrease of the subject's p38 mitogen-activated protein kinase 14 (p38MAPK) levels in the subject's cortex.

### BRIEF DESCRIPTION OF THE DRAWINGS

FIG. 1 shows the chemical structure of dihydroergotamine (DHE).

FIG. 2 shows the chemical structure of Sumatriptan.

FIG. 3 shows the chemical structure of 5-hydroxytryptamine (5-HT).

FIG. 4 shows a schematic of the mode of action of Sumatriptan (Suma) upon CGRP synthesis and secretion. SNAP: synaptosomal-associated protein(s); SNP: SNAP-interacting protein; NO: nitric oxide; CGRP: calcitonin gene-related peptide.

FIG. 5 shows the Sumatriptan repression of SNP- or SNAP-stimulated CGRP release.

FIG. 6 shows the effect of DHE or Sumatriptan on basal CGRP secretion levels.

FIG. 7 shows DHE or Sumatriptan repression of KCl-stimulated release.

FIG. 8 shows repression by DHE or Sumatriptan on capsaicin-stimulated release and that DHE does not significantly repress capsaicin-stimulated CGRP release.

FIG. 9 shows increase in MAP kinase phosphatase-1 (MKP-1) in DHE-treated trigeminal ganglia neurons.

FIG. 10 shows DHE-induced repression of p38 MAP kinase 14 levels in trigeminal ganglion neurons treated with vehicle (left panel), capsaicin (center panel), or capsaicin and DHE (right panel).

FIG. 11 shows decreased dye coupling (TRUEBLUE stain) between satellite glial cells and trigeminal ganglia neurons treated with either capsaicin (left panel) or with capsaicin and DHE (right panel).

FIG. 12 shows DHE-induced repression of connexin 26 levels in trigeminal ganglion neurons and satellite glia.

FIG. 13 shows expression of CGRP in cultured trigeminal ganglion neurons: Nomarski differential interference contrast (DIC) photomicrograph (left panel); β-tubulin (center panel); and CGRP (right panel). Bar equals 50 μm.

FIG. 14 shows expression of 5-HT₁ receptors in cultured trigeminal ganglion neurons: Row A: 5-HT_{1B}, 5-HT₁, 5-HT_{1F}, and 5-HT_{1B}/5-HT_{1D}/5-HT_{1F} co-stain; Row B: β-tubulin.

FIG. 15 shows DHE inhibits stimulatory effect of capsaicin on CGRP levels in trigeminal ganglion neurons treated in vivo. Upper row: CGRP stain; lower row: DAPI stain; left panels: control vehicle; center panels: capsaicin; right panels: capsaicin and DHE together.

FIG. 16 shows DHE increases expression of MAP kinase phosphatases (MKPs) in trigeminal ganglion neurons and satellite glial cell in vivo. Upper row: MKP stain; center row: DAPI stain; lower row: merged MKP/DAPI stain images; first panels: control vehicle and non-specific Ab; second panels: MKP-1 Ab; third panels: control vehicle and non-specific Ab; fourth panels: MKP-2 Ab; fifth panels: control vehicle and non-specific Ab; sixth panels: MKP-3 Ab.

FIG. 17 illustrates a scenario by which DHE can exert effects at multiple targets during the prodrome phase of migraine.

### DETAILED DESCRIPTION OF THE INVENTION

The embodiments disclosed in this document are illustrative and exemplary and are not meant to limit the invention. Other embodiments can be utilized and structural changes can be made without departing from the scope of the claims of the present invention.

As used herein and in the appended claims, the singular forms “a,” “an,” and “the” include plural reference unless the context clearly dictates otherwise. Thus, for example, a reference to “a particle” includes a plurality of such particles, and a reference to “a carrier” is a reference to one or more carriers and equivalents thereof, and so forth.

Definitions

“Migraine” and “migraine headache” is defined herein as a recurrent, throbbing headache generally, but not always, felt on one side of the head.

“Dihydroergotamine,” “DHE,” “dihydroergotamine mesylate” and synonyms thereof, shall be defined as a therapeutic amount of dihydroergotamine or a pharmaceutical acceptable derivative or salt thereof.

“Analgesic” shall be defined as a chemical substance capable of causing diminished sensitivity to pain.

“Antiemetic” shall be defined as a chemical substance capable of causing diminished nausea and or vomiting.

“Vasoconstrictor” shall be defined as a chemical substance that induces the narrowing of the lumen of blood vessels, i.e., vasoconstriction. “Non-vasodilating” shall be defined as a compound, drug, pharmaceutical, treatment or therapy that does not induce vasoconstriction.

“Therapeutic formulation” shall be described as a pharmaceutical composition comprising at least one active ingredient along with other optional ingredients useful in, for example, binding, flavoring, coloring, preserving, stabilizing, increasing shelf life, adding structural rigidity, adding desired mouth feel, adding desired mouth consistency, aiding in regulating dissolution rate, adjusting the pH of the local environment or adding adhesive qualities to promote absorption into the systemic circulation.

“Administered in combination”, “co-administered” or equivalent terms, shall be defined as pharmaceuticals that are administered simultaneously or sequentially with DHE. The pharmaceuticals administered need not be in the same dosage form (i.e., sublingual, in aerosol form, and/or) as the DHE. “In combination with” DHE shall be defined as the administration of the other drug either simultaneously or sequentially with DHE.

“pH adjusting agent” shall be defined as a compound that, alters or adjusts the pH of the local environment. In the context of the present invention, a “pH adjusting agent” alters or adjusts the pH of the sublingual area upon dissolving. The pH of DHE in solution is typically in the range of 3.2-4.0.

“Reduced” and “reduced symptoms” shall be defined as a lessening of symptoms to a noticeable degree by either the patient or medical professional. In the context of the present invention reduced symptoms shall mean, for example, the lessened severity of the subject's migraine headache. “Lessened severity” shall be defined, for example, as reduced pain, reduced throbbing, an increased ability for the subject to perform his or her normal routine, etc. It is not necessary, in the context of the present invention, for the treatment to relieve all symptoms of the migraine or to completely relieve the symptoms of the migraine.

“Subject” shall be defined as a person having symptoms of migraine headaches.

As used herein, the term “spray” refers to a liquid minutely divided or nebulized as by a jet of gas(es).

As used herein, the terms “aerosol” or “aerosolized” refer to a gaseous suspension of fine solid or liquid particles.

As used herein, the term “sol-gel” refers to a colloidal suspension which may transition from a liquid (sol) to a more solid material (gel).

The present invention is directed, among other things, to methods of treating migraine by treating a subject in the prodrome phase of a migraine with DHE, wherein DHE represses intracellular transport of inflammatory molecules via gap-junctions. In addition the invetion is drawn to a method for treating migraine wherein treatment using a pharmaceutical compound upregulates expression and activity of mitogen-activated protein kinase phosphatase (MAP kinase phosphatase) in the subject's nervous system.

Delivery of Pharmaceutical Compositions

An ergot alkaloid, such as DHE, alone or in combination with other suitable components, can also be made into aerosol formulations to be administered via a spray. These aerosol formulations can be placed into pressurized acceptable propellants, such as dichlorodifluoromethane, nitrogen, and the like. They may also be formulated as pharmaceuticals for non-pressured preparations such as in a nebulizer or an atomizer.

In one embodiment, DHE is administered as an aerosol or a suspension directly to the lung epithelium, for example, using a nebulizer, atomizer, spray dispenser, or the like. DHE may be administered to the either the alveolar epithelium, the bronchial epithelium, or both. In another embodiment, DHE is administered to the lung epithelium in the form of particles having a diameter of the range of about 0.05 to 20 μm. In a more preferred embodiment the particle diameter is of the range of between about 0.05 to 10 μm. In a yet ore preferred embodiment the particle diameter is of the range of between about 0.4 to 3 μm.

In a preferred embodiment, DHE is administered as a solution comprising about 0.01% to about 0.5% DHE. More preferably, the solution is a physiological saline solution. Preferably, the amount of solution administered is about 0.1 ml (0.5 mg) to about 5 ml @1 mg/ml, depending on, for example, the concentration of the active ingredient. More preferably, the amount of solution is about 2.5-5 ml and is delivered as a suspension using a metered dose inhaler.

The present disclosure describes novel, stable formulations of ergot alkaloid, such as, but not limited to dihydroergotamine (DHE), or pharmaceutically acceptable salts thereof, to administer dry powders and propellant suspensions via pulmonary aerosol inhalation or nasal spray inhalation. In one embodiment, DHE is used as the mesylate salt. The DHE powder is generated using a supercritical fluid processes. Supercritical fluid processes offer significant advantages in the production of DHE particles for inhalation delivery. Importantly, supercritical fluid processes produce respirable particles of the desired size in a single step, eliminating the need for secondary processes to reduce particle size. Therefore, the respirable particle produced using supercritical fluid processes have reduced surface free energy, which results in a decreased cohesive forces and reduced agglomeration. The particles produced also exhibit uniform size distribution. In addition, the particles produced have smooth surfaces and reproducible crystal structures which also tend to reduce agglomeration.

Such supercritical fluid processes may include rapid expansion (RES), solution enhanced diffusion (SEDS), gas-anti solvent (GAS), supercritical antisolvent (SAS), precipitation from gas-saturated solution (PGSS), precipitation with compressed antisolvent (PCA), aerosol solvent extraction system (ASES), or any combinations of the foregoing. The technology underlying each of these supercritical fluid processes is well known in the art and will not be repeated in this disclosure. In one specific embodiment, the supercritical fluid process used is the SEDS method as described by Palakodaty et al. in US Application 2003 0109421.

The supercritical fluid processes produce dry particulates that can be used directly by premetering into a dry powder inhaler (DPI) format, or the particulates may be suspended/dispersed directly into a suspending media, such as a pharmaceutically acceptable propellant, in a metered dose inhaler (MDI) format. The particles produced may be crystalline or may be amorphous depending on the supercritical fluid process used and the conditions employed (for example, the SEDS method is capable of producing amorphous particles). As discussed above, the particles produced have superior properties as compared to particles produced by traditional methods, including but not limited to, smooth, uniform surfaces, low energy, uniform particle size distribution and high purity. These characteristics enhance physicochemical stability of the particles and facilitate dispersion of the particles, when used in either DPI format or the MDI format.

The particle size should be such as to permit inhalation of the DHE particles into the lungs on administration of the aerosol particles. In one embodiment, the particle size distribution is less than 20 microns. In an alternate embodiment, the particle size distribution ranges from about 0.050 μm to 10.000 μm MMAD as measured by cascade impactors; in yet another alternate embodiment, the particle size distribution ranges from about and preferably between 0.4 and 3.5 μm MMAD as measured by cascade impactors.

The supercritical fluid processes discussed above produce particle sizes in the lower end of these ranges.

In the DPI format the DHE particles can be electrostatically, cryometrically, or traditionally metered into dosage forms as is known in the art. The DHE particle may be used alone (neat) or with one or more pharmaceutically acceptable excipients, such as carriers or dispersion powders including, but not limited to, lactose, mannose, maltose, etc., or surfactant coatings. In one preferred formulation, the DHE particles are used without additional excipients. One convenient dosage form commonly used in the art is the foil blister packs. In this embodiment, the DHE particles are metered into foil blister packs without additional excipients for use with a DPI. Typical doses metered can range from about 0.050 mg to 2 mg, or from about 0.250 mg to 0.500 mg. The blister packs are burst open and can be dispersed in the inhalation air by electrostatic, aerodynamic, or mechanical forces, or any combination thereof, as is known in the art. In one embodiment, more than 25% of the premetered dose will be delivered to the lungs upon inhalation; in an alternate embodiment, more 50% of the premetered dose will be delivered to the lungs upon inhalation; in yet another alternate embodiment, more than 80% of the premetered dose will be delivered to the lungs upon inhalation. The respirable fractions of DHE particles (as determined in accordance with the United States Pharmacopoeia, chapter 601) resulting from delivery in the DPI format range from 25% to 90%, with residual particles in the blister pack ranging from 5% or the premetered dose to 55% of the premetered dose.

In the MDI format the particles can be suspended/dispersed directly into a suspending media, such as a pharmaceutically acceptable propellant. In one particular embodiment, the suspending media is the propellant. It may be desirable that the propellant not serve as a solvent to the DHE particles. Suitable propellants include C₁₋₄ hydrofluoroalkane, such as, but not limited to 1,1,1,2-tetrafluoroethane (HFA 134a) and 1,1,1,2,3,3,3-heptafluoro-n-propane (HFA 227) either alone or in any combination. Carbon dioxide and alkanes, such as pentane, isopentane, butane, isobutane, propane and ethane, can also be used as propellants or blended with the C₁₋₄ hydrofluoroalkane propellants discussed above. In the case of blends, the propellant may contain from 0-25% of such carbon dioxide and 0-50% alkanes. In one embodiment, the DHE particulate dispersion is achieved without surfactants. In an alternate embodiment, the DHE particulate dispersion may contain surfactants if desired, with the surfactants present in mass ratios to the DHE ranging from 0.001 to 10. Typical surfactants include the oleates, stearates, myristates, alkylethers, alkylarylethers, sorbates and other surfactants used by those skilled in the art of formulating compounds for delivery by inhalation, or any combination of the foregoing. Specific surfactants include, but are not limited to, sorbitan monooleate (SPAN-80) and isopropyl myristate. The DHE particulate dispersion may also contain polar solvents in small amounts to aid in the solubilization of the surfactants, when used. Suitable polar compounds include C₂₋₆ alcohols and polyols, such as ethanol, isopropanol, polypropylene glycol and any combination of the foregoing. The polar compounds may be added at mass ratios to the propellant ranging from 0.0001% to 4%. Quantities of polar solvents in excess of 4% may react with the DHE or solubilize the DHE. In one particular embodiment, the polar compound is ethanol used at a mass ratio to the propellant from 0.0001 to 1%. No additional water or hydroxyl containing compounds are added to the DHE particle formulations other than is in equilibrium with pharmaceutically acceptable propellants and surfactants. The propellants and surfactants (if used) may be exposed to water of hydroxyl containing compounds prior to their use so that the water and hydroxyl containing compounds are at their equilibrium points. Standard metering valves (such as from Neotechnics, Valois, or Bespak) and canisters (such as from PressPart or Gemi) can be utilized as is appropriate for the propellant/surfactant composition. Canister fill volumes from 2.0 ml to 17 ml may be utilized to achieve dose counts from one (1) to several hundred actuations. A dose counter with lockout mechanism can optionally be provided to limit the specific dose count irrespective of the fill volume. The total mass of DHE in the propellant suspension will typically be in the range of 0.100 mg to 2.000 mg of DHE per 100 mcL of propellant.

An actuator with breath actuation can preferably be used to maximize inhalation coordination, but it is not mandatory to achieve therapeutic efficacy. The respirable fraction of such MDIs would range from 25% to 75% of the metered dose (as determined in accordance with the United States Pharmacopoeia, chapter 601).

Such methods for delivery of ergot alkaloids are well known to those of skill in the art and are described in, for example, US Patent Application Number 2008/0118442.

In one embodiment treatment with DHE results in a decrease of the subject's score by at least 5% percent compared with a baseline measurement made prior to administrating the pharmaceutical formulation, preferably by at least 10%, or at least 15% or at least 20%, and often by 30% or greater.

In one embodiment treatment with DHE results in a decrease in the subject's cortical levels of neuropeptide is decreased by at least 5 percent compared with a baseline measurement made prior to administrating the pharmaceutical formulation, preferably by at least 10%, or at least 15% or at least 20%, and often by 30% or greater.

In one embodiment treatment with DHE results in a decrease in the subject's circulating von Willebrand factor by at least 5 percent compared with a baseline measurement made prior to administrating the pharmaceutical formulation, preferably by at least 10%, or at least 15% or at least 20%, and often by 30% or greater.

In one embodiment treatment with DHE results wherein electrical activity in the brain of the subject is decreased by at least 5 percent compared with a baseline measurement made prior to administrating the pharmaceutical formulation, preferably by at least 10%, or at least 15% or at least 20%, and often by 30% or greater.

In one embodiment treatment with DHE results wherein transport of inflammatory molecules between neural tissues in the brain of the subject is decreased by at least 5 percent compared with a baseline measurement made prior to administrating the pharmaceutical formulation, preferably by at least 10%, or at least 15% or at least 20%, and often by 30% or greater.

In one embodiment treatment with DHE results in a decrease in the subject's cortical levels of neuropeptide is decreased by at least 5 percent compared with a baseline measurement made prior to administrating the pharmaceutical formulation, preferably by at least 10%, or at least 15% or at least 20%, and often by 30% or greater.

In one embodiment treatment with DHE results in a decrease in the subject's connexin 26 is decreased by at least 5 percent compared with a baseline measurement made prior to administrating the pharmaceutical formulation, preferably by at least 10%, or at least 15% or at least 20%, and often by 30% or greater.

In some instances, it may beneficial to the subject wherein any of the above differences in parameter levels are changed by at least 10%, by at least 15%, or at least 20%, or at least 25%, or at least 30%, or at least 35%, or at least 45%, or at least 50%, or at least 55%, or at least 65%, or greater.

DHE

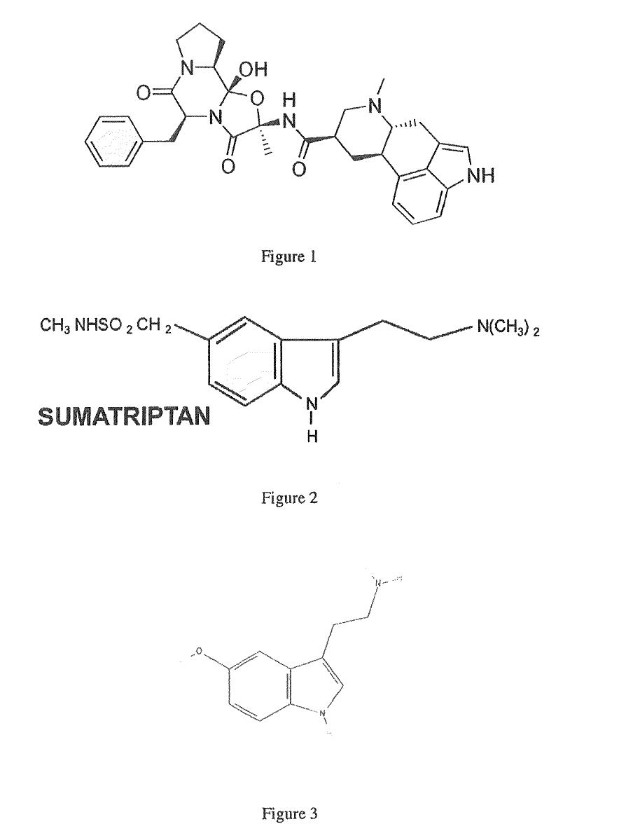

The chemical structure of DHE is shown in FIG. 1. Comparison with the structures of Sumatriptan (FIG. 2) and serotonin (5-HT; FIG. 3) suggests that they share a common site that interacts with the 5-HT₁ receptor. However, the effects of the other portions of the DHE structure may also interact with other as yet unknown cell-surface receptors, intracellular molecules, and even nuclear envelope components.

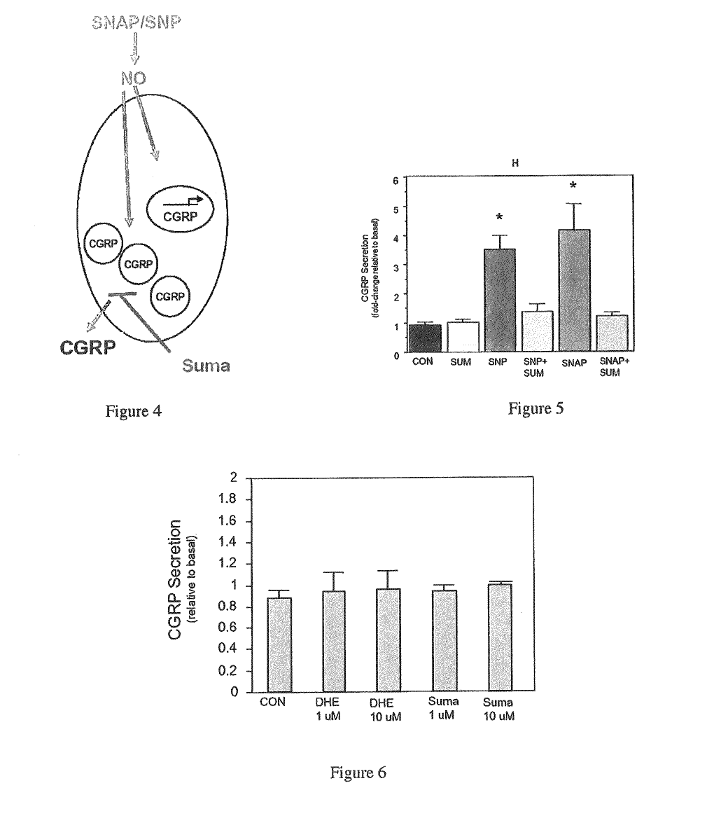

FIG. 4 shows a schematic diagram illustrating the potential mechanism by which Sumatriptan effects repression of CGRP secretion, probably via 5-HT₁ receptor-mediated signaling pathways. FIG. 5 illustrates the result of an experimental model of CSD induction in cell cultures. Treatment of cells with synaptosomal-associated protein(s) (SNAP), SNAP-interacting protein (SNP), induces the intracellular formation of nitric oxide (NO) that, in turn, induces transcription and translation of calcitonin gene-related peptide (CGRP) gene. Treatment with Sumatriptan (Suma) abolishes much of the induction.

FIG. 17 illustrates a schematic showing how DHE is proposed to act via the to pre-emptively repress CSD, the subsequent secretion of CGRP and the onset of migraine.

The following is an explanation of the various physiological pathways that DHE effects when administered during a migraine prodrome. The general functional pathway is shown in blue above.

Various triggering events (such as stress, dietary components, hormonal changes, infection with virus or other microbial pathogen) can initiate Cortical Spreading Depression (CSD), a proposed initiating event for migraine pain, which results in the release of CGRP, kinins, and Substance P from the glia and endothelial cells. When these neurotransmitters effect the trigeminal nerve they cause pain and a second release of CGRP.

Conventional prophylaxis usually entails the regular and repetitive administration of various over-the-counter and prescription medication such as Sumatriptan which results in a 50% decrease in migraine attacks in 50% of the persons under the therapy.

DHE, when administered during prodrome, exerts its action via three mechanisms indicated in the red numbers in FIG. 17.

#1—By interfering with the stimulus of the trigging event there is no CSD and thus no migraine pain.

#2. Even if a CSD occurs, DHE interferes with the resulting production of CGRPs, kinins, and Substance P.

#3. Further DHE interferes with the release of CGRPs from the trigeminal nerves.

DHE Treatment of Cell Cultures in vitro

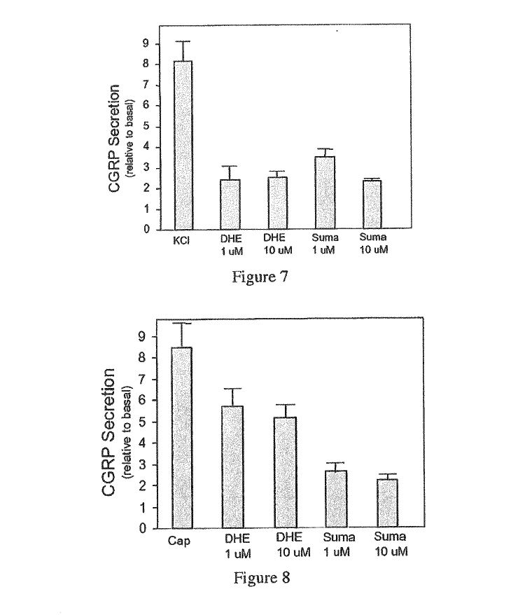

DHE has been shown to particularly repress expression of CGRP, an inflammatory molecule produced by glia and neurons that can increase vasodilation of proximal blood vessels. As shown in FIG. 7, DHE represses release (secretion) of CGRP from the cells stimulated by KCl.

DHE Treatment of Subject in vivo

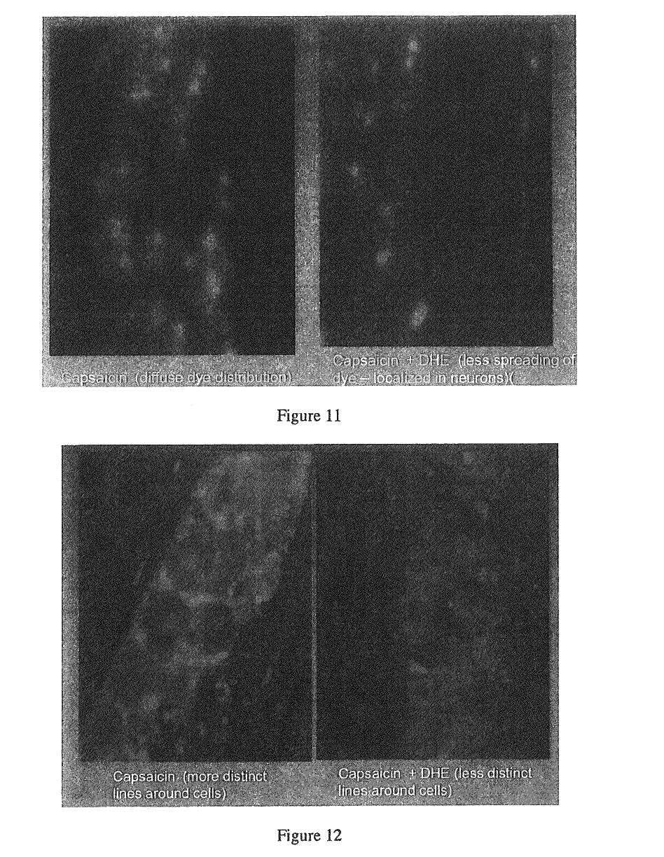

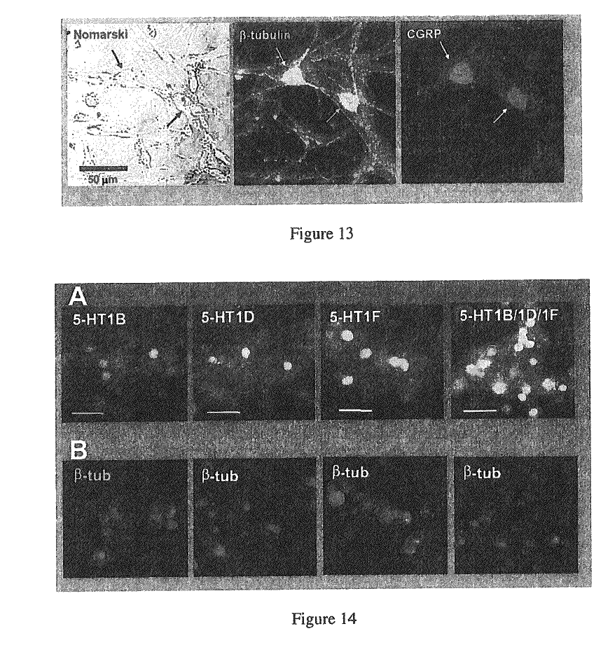

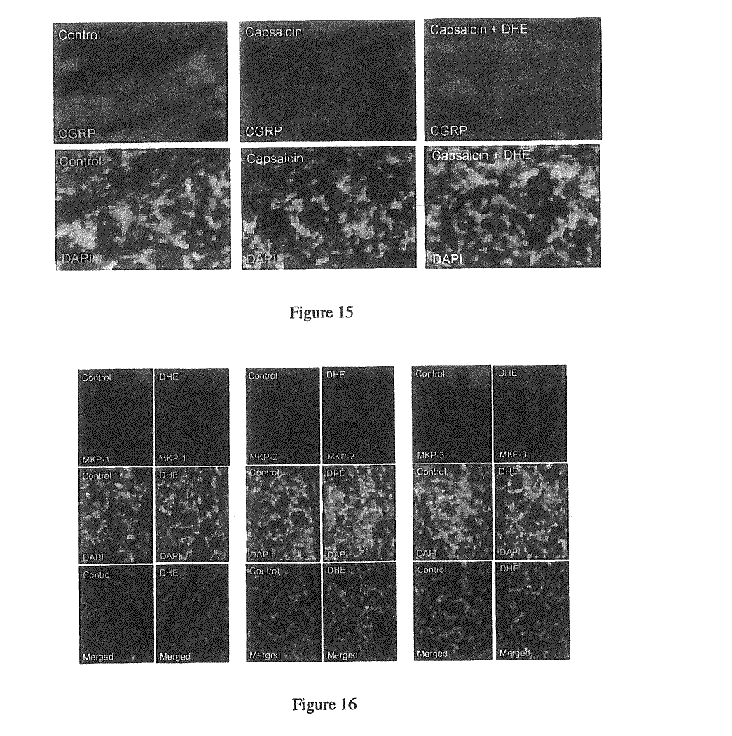

We have now found that, surprisingly, DHE appears to block inter-cellular transport via gap-junctions, in particular, perhaps by (i) binding to the gap junction complex, thereby blocking the channel, (ii) by blocking translation/transcription of new connexin 26, a component of gap-junctions, thereby reducing the number of potential gap junctions that may be created, (iii) both mechanisms (i) and (ii), or (iv) by another mechanism that involves 5-HT₁ receptor interactions with gap junction formation/activation, via additional signal transduction pathway(s). As shown in FIGS. 11 and 12, DHE represses diffusion of TRUEBLUE dye between trigeminal ganglial cells and decreases the levels of connexin 26 in the cell surface membranes those cells.

This also suggested that recruitment of connexin 26 to the gap junction might also be modulated by DHE and that upstream regulators of connexin induction might be affected or acted upon by DHE.

This disruption in neuronal communication or transmission between trigeminal neurons and glial cells is likely to reduce migraine severity, associated side effects and recurrence.

Modulation of MAP Kinase Phosphatase Activity and Upstream Effectors

Activation of transport activity through gap junctions may be mediated by phosphorylation of connexin at tyrosine and serine/threonine residues by a number of protein kinases, including, but not limited to, casein kinase 1, c-SRC, MAP kinases ERK5 and ERK1/2, as well as the presence of increased intracellular [Ca²⁺]_{I}. These pathways are in turn activated by, for example, inflammatory cytokine (or lysophosphatidic acid) binding to receptors having tyrosine kinase activity that proceed to induce a cascade of further tyrosine kinase activities that activate downstream mixed tyrosine kinase and serine/threonine protein kinases such as MAPKKK and MAPKK. In contrast, the majority of neurotransmitters, such as serotonin, glutamate, dopamine, and noradrenalin, act via GPCRs and follow only serine/threonine protein kinase pathways, such as PK-A, PK-C. Interestingly, NO, that induces expression of CGRP, acts via another serine/threonine kinase, PK-G.

One modulator of MAP kinase activity is MAP kinase phosphatase, that is known to dephosphorylate MAP kinase, thereby inactivating the enzyme; this would result in reducing phosphorylation of connexins and result in reduced gap junction formation.

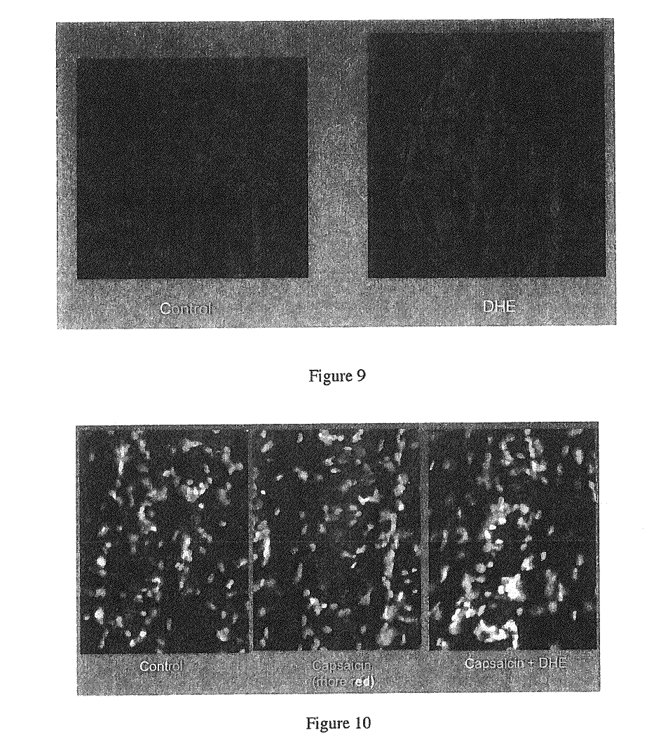

We therefore monitored MAP kinase phosphatase levels in control, treated with capsaicin, and treated with DHE and capsaicin together. The results, as shown in FIGS. 9, 10, and 16, suggest that DHE modulates the MAP kinase signal transduction pathway by increasing MAP kinase phosphatase-1 (MKP-1), MKP-2, and MKP-3 levels (and activity) as well as repressing p38 MAP kinase 14 levels following stimulation by capsaicin.

We therefore concluded that treatment of a subject having a prodrome phase of migraine with DHE has a protective effect upon the subject's neural, glial, and endothelial tissue via induction of MAP kinase phosphatase activity. This effect can be acute or it can be long-term, or the effect can be both acute and long-term. Any other compond that has a similar protective effect will also be useful in the treatment of a migraineur, and, in the long-term, reduce the risk of a future stroke.

Gallagher et al. have recently suggested that the MAP kinase/phosphatase pathway is a primary molecular mechanism for regulating ACE2 to maintain the balance between Ang II and Ang-(1-7). Angiotensin converting enzyme 2 (ACE2) catalyzes the conversion of the vasoconstrictor angiotensin II (Ang II) to the vasodilatory peptide angiotensin-(1-7) [Ang-(1-7)]. Down-regulation of ACE2 was shown to be modulated by MAP kinase phosphatase thereby reducing the rate of conversion of Ang II to Ang-(1-7). (Gallerher et al. (2008) Am. J. Physiol. Cell Physiol. September 3 [Epub ahead of print]).

In addition, Cho et al. (2008) have shown that toll-like receptor-4 ligand activates transcription of MAP kinase phosphatase via (C/EBP)β transcription factor, thereby activating a long-term modulating effect upon MAP kinase. Of note, binding of (C/EBP)β transcription factor to the promoter of the MAP kinase phosphatase gene is also induced by glucocorticoids (Cho et al. (2008) Arch. Biochem. Biophys. August 19 [Epub ahead of print]; Johansson Haque (2008) J. Mol. Endocrinol. August 5 [Epub ahead of print]).

In contrast, in cells exposed to heat shock, MAP kinase phosphatase is inactivated, probably by aggregation with the heat shock protein Hsp72. This provides another mechanism by which MAP kinase phosphatase may be regulated in vivo. (Yaglom et al. (2003) Mol. Cell. Biol. 23(11): 3813-3824.)

There are therefore many different molecular mechanisms that may activate MAP kinase phosphatase pathways, each of which may be used in a pharmaceutical formulation as therapy for migraine and related headache. For example, neurotransmitters such as acetylcholine, adrenaline, noradrenaline, glutamate, aspartate, glycine, taurine, dopamine, 5-hydroxytryptamine, melatonin, y-aminobutyric acid, histamine, bradykinin, endorphins, enkephalins, a-MSH, neurokinins, oxytocin, substance P, as well as glucocorticoids, and toll-like receptor-4 ligand, and an ACE inhibitor, any derivatives and metabolites thereof, and the like.

Electro-Magnetic Stimulation

Migraine patients are postulated to have different ability in processing pain and other sensations compared to non-migraine patients, that may account for symptoms such as, for example, photophobia, phonophobia, and the like. One such difference is believed to be a hyperexcitable state of their cerebral cortex compared to normal people. There are several ways of measuring the excitable state of the cerebral cortex. One method, which appears to be simple to perform in appropriately equipped and experienced lab and is consistently reproducible, uses electromagnetic stimulation of the cerebral cortex.

Electromagnetic stimulation tends to suppress (inhibit) cortical activity at a certain frequency. Such suppression can be measured by observing the rate at which an individual can read and comprehend a set of three letters or number presented for a very brief period of time. In principle, the higher the suppression of cortical activity, the more mistakes the individual will record. Such mistakes are usually reciting a set of letters or numbers in another sequence and/or replacing a letter or number with another. In a non-migranous patient the mistakes made are far more frequent compared to those made by a migraineur, suggesting that it is difficult to suppress cortical activity in migraineurs because of the hyper-excitable state of the brain. There appears to be an inverse correlation between the number of mistakes an individual makes during the stimulation and the degree of migraine severity. Also treatment with prophylactic medications appears to increase the frequency of mistakes, suggesting return to normalcy. There also is a correlation between the rate/degree of return to normalcy and the efficacy of the prophylactic medicine used to treat the migraine.

Thus, the degree of cortical suppression induced by electromagnetic stimulation can be used to compare the potential efficacy of different drugs in preventing migraine attacks.

Migraine pain Intensity in Subject as Proxy for Efficacy of DHE Treatment

In order to evaluate the efficacy of pre-emptive treatment for a migraine, a subject is be administered one or both of the following two protocols that evaluate migraine frequency and the intensity levels of pain as experienced by the subject.

Functional evaluations include (i) HEADACHE IMPACT TEST (HIT). HIT provides an accurate description of the impact headaches are having on daily life and (ii) the Migraine Disability Assessment test (MIDAS) questionnaire that measures the impact of headaches on life over the last 3 months. Pain Scoring may be performed as follows: IHS Pain Intensity Scoring (Pain Free=0, Mild=1, Moderate=2, Severe=3) In addition, the Allodynia Symptom Checklist (ASC) probes severity of cutaneous allodynia. Furthermore, the Migraine Assessment of Current Therapy (Migraine-ACT) questionnaire probes four clinically important domains of the migraine experience are assessed: consistency of response, global assessment of relief, headache impact, and emotional response.

Subjects may be evaluated before, during and following treatment with DHE on an outpatient basis and the results may be used by a clinician to continuously evaluate the subsect's frequency of migraine episodes, as well as to monitor any long-term effects that may become apparent.

Cortical Spreading Depression Animal Model

It is believed that an excitatory, epilepsy-like, electrical wave spreading across the cerebral cortex, commonly known as cortical spreading depression (CSD), is a precursor of an acute migraine attack. CSD initiates a cascade of chemical and electrical changes (release of CGRP, neurokinins, prostaglandins, other inflammatory molecules, and vasodilatory effectors, etc.) that ultimately result in the headache pain. Any drug that suppresses or prevents CSD can prevent or reduce the frequency of headaches in migraine patients.

An experimental CSD can be induced in animals by injecting KCl or other irritants in the occipital cortex of the animal. The resulting CSD can be recorded and quantified using special and sophisticated recording devises. Using such a model one can compare the efficacy of different prophylactic drugs as to their ability in suppressing the number and severity of CSD.

Also using some of the recently developed animal models, the downstream effects of the CSD—that is, the amount of CGRP released, inflammatory changes, c-fos gene activity in the trigeminal neuronal cells (TNC), etc., can also me measured and quantified using methods well-known to those of skill in the art.

Treatment of Other Conditions

It has been observed that the risk for stroke (cerebral ischaemia) appears to be greater in migraineures than in the non-migraineur population (Tietjen (2000) Neuroepidemiology 19(1): 13-19;). This can be explained by the knowledge that repeated vasodilation of the blood vessels in the cerebral cortex and the dura results in increased endothelial synthesis and secretion into the blood of von Willebrand factor (vWf), a known risk factor for the cerebral ischaemia and related conditions and disorders (see, for example, Tietjen (2000) Neuroepidemiology 19(1): 13-19; McGirt et al. (2002) Neurosurgery 51(5): 1128-1134; Tietjen (2005) CNS Drugs 19(8): 683-692; Frijns et al. (2006) J. Neurol. Neurosurg. Psychiatry 77(1): 77-83; ibid (2006) J. Neurol. Neurosurg. Psychiatry 77(7): 863-867).

As noted herein, it has been speculated that subjects having a higher incidence and susceptibility to having migraines are also at a greater risk for stroke and perhaps other disorders in which the brain tissue is subject to insult from blood products, such as mini-strokes, that may predispose an individual to dementias. Reducing the incidence of CSD may well have long-term effects upon both the individual (less likely to suffer a debilitating stroke) and society (less future catastrophic health care expenditures).

The invention will be more readily understood by reference to the following examples, which are included merely for purposes of illustration of certain aspects and embodiments of the present invention and not as limitations.

### EXAMPLES

### Example I

### DHE Suppresses Secretion of Inflammatory Molecules in vitro

These experiments investigated the cellular events within trigeminal ganglia that may account for the therapeutic benefit of DHE in the pre-emptive treatment of migraine and cluster headache.

Trigeminal ganglia comprise ˜10% neurons, ˜90% glia, and ˜2% Schwann cells. They are located in the mammalian head, usually posterior and adjacent to the orbit. Primary trigeminal ganglion cultures were established using trigeminal ganglia dissected from day 2-3 (2-3PN) neonate Sprague Dawley rats. Cultures were maintained for 1 d and were then untreated (control), treated 1 h with 60 mM KCl, 1 h with 2 μM capsaicin, 1 h with 1 μM or 10 μM DHE, 1 h with 1 μM or 10 μM Sumatriptan, or pretreated with DHE or Sumatriptan for 30 minutes prior to addition of stimulatory agents.

The amount of CGRP released into the culture medium was determined by radioimmunoassay and normalized to total protein as determined using the modified method of Bradford (Bradford (1976) Anal. Biochem. 72: 248-254). Statistical significance was determined using Mann-Whitney U non-parametric test. Differences considered statistically significant at p<0.05. Cultured cells were also stained for protein expression of β-tubulin, CGRP, and 5-HT₁ receptors using specific antibodies (Abs) and immunohistochemistry.

FIG. 6 shows that DHE or Sumatriptan (Suma) had no apparent effect upon basal secretion of CGRP into the medium. However, stimulation of the culture using KCl was reduced in the presence of DHE and Suma by approximately 68% and 70%, respectively (see FIG. 7). In addition, stimulation of the culture using capsaicin was reduced in the presence of DHE and Suma by approximately 38% and 71%, respectively (see FIG. 8).

FIGS. 14A and 14B show typical results for immunohistochemical staining using Abs against β-tubulin, CGRP, and 5-HT₁ receptors. The results show that the expression of CGRP and 5-HT₁ receptors co-localized with the cells and with β-tubulin.

### Example II

### DHE Suppresses Secretion of Inflammatory Molecules in vivo

Adult (A) Sprague Dawley rats were anaesthetized by intraperitoneal (i.p.) injection of 0.3 ml ketamine and xylazine (Sigma Chemical Co. St. Louis, Mo.; 800 mg and 60 mg per 10 ml, respectively). The animals were then injected in the eyebrow region with 10 μM capsaicin for 2 h, 10 mg.kg⁻¹ DHE i.p. for 1 h, or were pretreated with DHE for 1 h prior to injection with capsaicin. Trigeminal ganglia were collected and placed in optimal cutting temperature (OCT) prior to cryosectioning. Sections were then stained using antibodies for CGRP and MKPs.

As shown in FIG. 9, treatment with DHE resulted in an increase of MAP kinase phosphate-1 levels by at least 10% in the trigeminal ganglial neurones and satellite glia. Figure G shows that similar results were obtained in separate experiments to determine levels of MKP-1, MKP-2, and MKP-3 following treatment with DHE. FIG. 10 shows that treatment with DHE also repressed capsaicin-induced expression of p38 MAP kinase 14.

FIG. 11, in contrast, shows that DHE repressed capsaicin-induced diffusion of TRUEBLUE dye between neurons and glia at least 10%%. FIG. 12 shows that levels of connexin 26, a gap junction component protein, are also repressed following treatment with DHE.

### Example III

### Immunohistochemistry

Primary trigeminal ganglion cultures or 20 μm sections of trigeminal ganglia were fixed in 4% paraformaldehyde, stained with antibodies for CGRP (Neuromics, 1:500), β-tubulin (Sigma, 1:1000), 5-HT₁ receptors (Santa Cruz, 1:100), MKP-1 (Upstate, 1:500), MKP-2 (Santa Cruz, 1:500), or MKP-3 (Santa Cruz, 1:500). Immunoreactive proteins were visualized using rhodamine Red-X-conjugated (β-tubulin and MKPs) or FITC-conjugated (5-HT₁ and CGRP) secondary antibodies (1:100 dilution in PBS, Jackson ImmunoResearch Laboratories).

### Example IV

### Perception of Migraine Pain is Reduced following Pre-Emptive Treatment with DHE

In order to evaluate the efficacy of pre-emptive treatment for a migraine, a subject is administered one or both of the following two protocols that evaluate migraine frequency and the intensity levels of pain as experienced by the subject. A pharmaceutical composition is considered efficacious if the subject's experience of pain is significantly decreased following treatment with the pharmaceutical composition.

Functional Evaluations

HEADACHE IMPACT TEST (HIT). HIT provides an accurate description of the impact headaches are having on daily life.

The Migraine Disability Assessment test (MIDAS): Questionnaire that measures the impact of headaches on life over the last 3 months.

Pain Scoring

IHS Pain Intensity Scoring (Pain Free=0, Mild=1, Moderate=2, Severe=3) Allodynia Symptom Checklist (ASC) probes severity of cutaneous allodynia Migraine Assessment of Current Therapy (Migraine-ACT): Questionnaire that probes four clinically important domains of the migraine experience are assessed: consistency of response, global assessment of relief, headache impact, and emotional response.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

We claim:

1. A method for the prevention of migraine in a migraineur, the method comprising administering a pharmaceutical formulation directly to the lung epithelium by pulmonary aerosol inhalation to a subject during a migraine prodrome episode, the pharmaceutical formulation consisting of dihydroergotamine and a carrier, wherein said pharmaceutical is effective to prevent migraine or decrease migraine frequency or intensity levels of pain in the subject.

2. The method as set forth in claim 1 wherein the dihydroergotamine is the mesylate salt.

3. A method for the prevention of migraine in a migraineur, the method consisting of administering a pharmaceutical formulation by pulmonary aerosol inhalation to a subject during a migraine prodrome episode, the pharmaceutical formulation consisting essentially of dihydroergotamine and a propellant, wherein said administration is effective to prevent migraine or decrease migraine frequency or intensity levels of pain in the subject.

4. The method of claim 3, wherein the propellant is selected from the group consisting of 1,1,1,2-tetrafluoroethane (HFA 134a) and 1,1,1,2,3,3,3-heptafluoro-n-propane (HFA 227).

5. The method of claim 3, wherein the propellant is a mixture of 1,1,1,2-tetrafluoroethane (HFA 134a) and 1,1,1,2,3,3,3-heptafluoro-n-propane (HFA 227).

6. The method of claim 3, wherein pharmaceutical formulation is administered by pulmonary aerosol inhalation using a pressurized metered dose inhaler.
